(19) United States
(12) Patent Application Publication
(10) Pub. No.: US 2011/0269807 A1
(43) Pub. Date: Nov. 3, 2011

(54) NOVEL TREATMENT FOR AGE RELATED MACULAR DEGENERATION AND OCULAR ISCHEMIC DISEASE ASSOCIATED WITH COMPLEMENT ACTIVATION BY TARGETING 5-LIPOXYGENASE

(75) Inventor: Peter C. Baciu, Laguna Niguel, CA (US)

(73) Assignee: ALLERGAN, INC., Irvine, CA (US)

(21) Appl. No.: 13/098,200

(22) Filed: Apr. 29, 2011

Related U.S. Application Data
(60) Provisional application No. 61/330,147, filed on Apr. 30, 2010.
(60) Provisional application No. 61/357,416, filed on Jun. 22, 2010.

(30) Foreign Application Priority Data
Apr. 29, 2011 (US) PCT/US11/34599

(51) Int. Cl. A61K 31/4155; A61P 27/02; A61K 31/405; A61K 31/381; A61K 31/05

(52) U.S. Cl. 514/406; 514/443; 514/734; 514/418

(57) ABSTRACT

The invention relates to compounds, compositions, drug delivery systems, and methods for treating age-related macular degeneration (AMD) and ocular ischemic disease in an individual in need.

### CROSS-REFERENCE TO RELATED APPLICATIONS

This application claims the benefit to U.S. Provisional Application 61/330,147, filed Apr. 30, 2010, and U.S. Provisional Application 61/357,416, filed Jun. 22, 2010. Provisional Applications 61/330,147 and 61/357,416 are hereby incorporated by reference.

### BACKGROUND OF THE INVENTION

Age related macular degeneration, or AMD, is the leading cause of blindness The seminal characteristic of AMD is progressive loss of central vision attributable to degenerative and neovascular changes in the macula, a specialized area in the center of the retina. There are two forms of AMD, atrophic or dry AMD and neovascular or wet AMD. Typically AMD begins as dry AMD. Dry AMD is characterized by the formation of yellow plaque like deposits called drusen in the macula, between the retinal pigment epithelium (RPE) and the underlying choroid. About 15% of dry AMD patients develop wet AMD which is characterized by choroidal neovascularization, that is by the formation of new blood vessels in the subretinal space. Accordingly, effective treatments for AMD are desirable.

Among the many causative factors associated with age related macular degeneration, complement activation and oxidative stress are the most highly recognized. In the former a clear genetic linkage has been established, while in the latter the use of anti-oxidants have been demonstrated to be beneficial but not curative. Current therapeutic approaches rely on targeting one or the other pathways.

However, what had previously been unobvious is the synergistic relationship between oxidative stress and complement that drives the pathology. To address the potential synergy and identify novel therapeutic interventions driven by the synergy of oxidative stress and complement activation, we have developed an assay and used this assay to screen a diverse compound library to identify novel therapeutics. We developed a complement mediated cell death assay directed against RPE cells using the alternative complement activation pathway in the context of oxidative stress. We then used this assay to screen the NIH clinical library. We have identified the 5-lipoxygenase pathway as a key therapeutic target in blocking the synergy between complement and oxidative stress.

Arachidonate 5-lipoxygenase (5-lipoxygenase, 5-LO or Alox5) is a member of the lipoxygenase family of enzymes and increases with aging. (Uz T et al. Aging-associated up-regulation of neuronal 5-lipoxygenase expression: putative role in neuronal vulnerability. FASEB J 1998, 12:439-49; Manev H et al. Putative role of neuronal 5-lipoxygenase in an aging brain. FASEB J 2000, 14: 1464-69.) 5-LO converts arachidonic acid into the powerful inflammatory leukotrienes. Leukotrienes promote cancer, damage the brain, promote asthma, arthritis, psoriasis and ulcerative colitis (Steele V E et al. Lipoxygenase inhibitors as potential cancer chemopreventives, Cancer Epidemiol Biomarkers Prev 1999, 8:467-83). They may also promote atherosclerosis (Rasmark O. Editorial: 5-lipoxygenase-derived leukotrienes. Mediators also of atherosclerotic inflammation. Arterioscler Thromb Vasc Biol 2003, 23:1140-42. 57. Kiecolt-Glaser J K, et al. Chronic stress and age-related increases in the proinflammatory cytokine IL-6. Proc Natl Acad Sci USA 2003, 100:9090-95.) Only cells which contain both 5-lipoxygenase (5-LO) and 5-lipoxygenase-activating protein (FLAP) will produce leukotrienes (Dixon et al. (1990) Nature 343:282-284; and Reid et al. (1990) J. Biol. Chem. 265:19818-19823.

The 5-lipoxygenase enzyme is activated by glucocorticoids (cortisol), (Manev H et al. Putative role of neuronal 5-lipoxygenase in an aging brain. FASEB J 2000, 14: 1464-69) and inhibited by melatonin. Unfortunately, aging and interleukin-6 increase cortisol in humans, (Dean W. Adaptive homeostat dysfunction. Vit Res News 2005, 19(2):1-5), while melatonin decreases drastically with aging, (Dean W. Neuroendocrine theory of aging: an introduction. Vit Res News 2005, 19(1):1-4.)

This invention provides a method of treating age related macular degeneration and ocular ischemic disease associated with complement activation by targeting 5-lipoxygenase.

### BRIEF DESCRIPTION OF THE DRAWINGS

FIG. 1 shows that Anti-ARPE-19 serum leads to complement activation. (A) S-58 (Solid line) and S-60 (dotted line) polyclonal serum recognize ARPE-19 cells as determined by FACS analysis. S-58 Induces complement activation as determined by generation of C3a (B) and Soluble C5b-9 (C). S-58 induction of C5b-9 deposition on surface of ARPE19 cells as determined by FACS (D), S-58 (Solid line) S-60 (Dotted line)

FIG. 2 shows that complement attack on ARPE-19 cells leads to cell swelling (A), calcium influx (B) (a=55%, b=36%, c=24%, d=16%, and e=12%, f=55% S-58 in absence of serum and g=serum alone). Dose dependent cell death and ATP release (C). A functional alternative complement cascade is required for S-58 induced complement mediated cell death (D).

FIG. 3 shows that oxidative stress induced by H₂O₂ or t-BH leads to enhanced cell death (A) although this increase in cell death does not correlate with altered expression of cRegs on the cell surface (B).

FIG. 4 shows a scatter plot of results from library screen.

FIG. 5 shows examples of compounds with protective properties.

FIG. 6 shows that the 5-lipoxygenase activating protein (FLAP) inhibitor MK886 protects ARPE-19 cells from oxidative stress and complement attack

FIG. 7 shows dose dependent protection of ARPE-19 cells against cell death by the lipoxygenase inhibitor 2-TEDC.

FIG. 8 shows The 15 lipoxygenase inhibitor PD146176 has no protective activity in blocking cell death mediated by the synergy between complement attach and oxidative stress.

FIG. 9 shows that minocyclin dose dependently protects ARPE-19 cells against cell death mediated by the synergy between complement attack and oxidative stress.

FIG. 10 shows that the 5-lipoxygenase inhibitor PF-4191834 protected against functional loss of retinal ERG A and B-wave amplitudes (FIGS. 10 A and B, respectively) in animals in response to oxidative stress induced by intravitreal PQ injection. (* p<0.05 in student tTest)

FIG. 11 shows that the 5-lipoxygenase inhibitor PF-4191834 protected against functional loss of retinal ERG A-wave amplitudes in animals in response to blue light induced retinal damage. (* p<0.05 in student tTest)

FIG. 12 shows the scoring template used for evaluation of retinal damage in animals in response to light induced damage.

FIG. 13 shows a comparison of retinal structure and outer nuclear layer thickness in vehicle and 5-lox inhibitor treated animals after blue light induced retinal damage. Note, that in animals receiving 5-lox inhibitor have a higher level of protection in ONL thickness relative to vehicle treated animals.

### DETAILED DESCRIPTION OF THE INVENTION

Compounds, compositions, and methods of using such compounds and compositions for treating age-related macular degeneration (AMD) and ocular ischemic disease in a subject in need are described herein. The subject in need can be a human or non-human animal, including a mouse, rat, rabbit, or non-human primate, or any other non-human mammal suffering from or in need of treatment of AMD or ocular ischemic disease.

One embodiment of the present invention is a method for treating AMD or ocular ischemic disease in a subject in need thereof comprising administering to said subject a therapeutically effective amount of a compound that reduces 5-lipoxygenase (5-LO) activity.

Another embodiment of the present invention is a method for treating AMD or ocular ischemic disease in a subject in need thereof comprising administering to said subject a therapeutically effective amount of a compound that reduces 5-lipoxygenase (5-LO) activity by inhibiting 5-lipoxygenase activating protein (FLAP).

Accordingly, in certain embodiments of the present method the compound that reduces 5-lipoxygenase activity is one that targets the 5-lipoxygenase polypeptide or a gene encoding the 5-LO polypeptide, thereby inhibiting or reducing the activity of the 5-LO polypeptide. In accordance with this embodiment, the compound is one that selectively inhibits 5-lipoxygenase. Examples of compounds that inhibit the expression of a polypeptide, and thereby the activity of a polypeptide, include siRNAs (short interfering RNAs, double stranded RNAs of 21-23 nucleotides in length), antisense nucleic acids, and ribozymes.

In one embodiment the compound that selectively inhibits 5-lipoxygenase is PF-4191834 (Masferrer et al. (2010) J. Pharm, Experimental Therapeutics 334(1):294-301). In another embodiment the compound that directly or selectively inhibits 5-lipoxygenase and reduces 5-lipoxygenase activity is Zileuton.

In other embodiments the method comprises administering a compound that inhibits or reduces 5-lipoxygenase activity by inhibiting 5-lipoxygenase activating protein (FLAP) in an amount effective for treating AMD or ocular ischemic disease. The compound that selectively inhibits FLAP may be a small molecule, an antibody, an aptamer, an antisense nucleic acid, or a small interfering RNA (siRNA). In one example, the compound that inhibits FLAP is MK886.

Description of Compounds for Use in the Present Methods

Compounds for use in the present methods of treating age-related macular degeneration (AMD) and ocular ischemic disease can be any substance that selectively inhibits or reduces the expression, function, or activity of 5-lipoxygenase (5-LO) in the eye of an individual. Exemplary compounds that may be used to inhibit the expression or activity of 5-lipoxygenase (“inhibitors of 5-lipoxygenase”) include short interfering double stranded RNAs (siRNAs) directed to the gene encoding 5-lipoxygenase, single stranded antisense nucleic acids targeted to the gene encoding 5-lipoxygenase, or ribozymes targeted to the gene encoding 5-lipoxygenase, each of which can be used to reduce the expression of 5-LO in the eye of an individual in need. Alternatively, or in addition, the practitioner can use monoclonal or polyclonal antibodies or aptamers targeted to 5-LO, or small molecule compounds that selectively or directly inhibit the activity of the 5-LO enzyme in the eye of an individual in need. Alternatively, the practitioner can selectively reduce 5-LO activity by administering a compound that inhibits 5-lipoxygenase activating protein (FLAP).

Methods and materials for making and using short interfering RNAs to reduce the expression of a messenger RNA (mRNA), such as that encoding 5-lipoxygenase or FLAP, in a sequence specific manner are known in the art. See, for example, U.S. Pat. No. 7,056,704, which is hereby incorporated by reference. See also Elbashir et al. (2001) Nature 411 (6836):494-8. Short interfering RNAs, sometimes referred to as small interfering RNAs, or RNAi compounds, are designed to hybridize to the nucleotide sequence of the mRNA target. Exemplary nucleotide sequences of mRNAs encoding human 5-LO and FLAP are known, as shown by Table 1, below. The nucleotide and polypeptide sequences defined by the GenBank Accession numbers given below in Table 1 are hereby incorporated by reference. See also Table 1 in U.S. Pat. No. 7,829,535, which is hereby incorporated by reference, describing 5-LO and FLAP mRNAs from other mammalian species such as Rat and Mouse.

TABLE 1
GenBank Accession Numbers of exemplary mRNA and Polypeptide
Sequences for arachidonate 5-lipoxygenase, also known as
5-lipoxygenase, or 5-LO.

| | mRNA | | Protein | |
|---|---|---|---|---|
| Human | NM_000698 | Homo sapiens | NP_000689 | arachidonate 5- |
| 5-LO | GI: | arachidonate 5- | GI: 4502057 | lipoxygenase |
| | 62912458 | lipoxygenase | Version: Apr. | [Homo |
| | Version: | (ALOX5), | 24, 2011 | sapiens]. |
| | Apr. 24, | mRNA. | | |
| | 2011 | | | |
| Human | NM_001629 | Homo sapiens | NP_001620 | arachidonate 5- |
| FLAP | GI | arachidonate 5- | GI 15718675 | lipoxygenase- |
| | 324711027 | lipoxygenase- | Version Apr. | activating |
| | Version: | activating | 17, 2011 | protein isoform |
| | Apr. 17, | protein | | 1 [Homo |
| | 2011 | (ALOX5AP), | | sapiens]. |
| | | transcript | | |
| | | variant 1, | | |
| | | mRNA. | | |

Small molecule compounds for reducing 5-lipoxygenase activity are known in the art. For example, U.S. Pat. No. 6,063,928, which is hereby incorporated by reference, describes small molecules and methods for making such molecules and pharmaceutically acceptable salts thereof having the ability to inhibit 5-lipoxygenase enzyme. More specifically see the compounds described in column 1 through line 2 of column 3 of U.S. Pat. No. 6,063,928, hereby incorporated by reference. See also columns 1 and 2 and claims 1-11 in U.S. Pat. No. 5,883,106, which is hereby incorporated by reference. See also US Patent Application Publication 2008/0125474, by Graneto et al., especially paragraphs 0008-0013, showing the formulas of particular pyrazole analogs that can be used to selectively inhibit 5-LO in an individual. Thus, for example, the compound for inhibiting 5-lipoxygenase can have the formula:

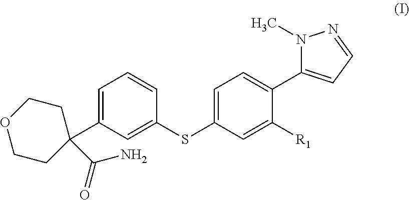

where R₁ is hydrogen (H) or fluorine (F).

More specifically the compound can be PF-4191834, having Formula I, above, where R₁ is hydrogen (H). PF-4191834 is a selective non-redox 5-lipoxygenase inhibitor and is described in Masferrer, J. L., et al., Pharmacology of PF-4191834), a selective non-redox 5-lipoxygenase inhibitor. Effective in inflammation and pain. J Pharmacol Exp Ther. 334(1): p. 294-301

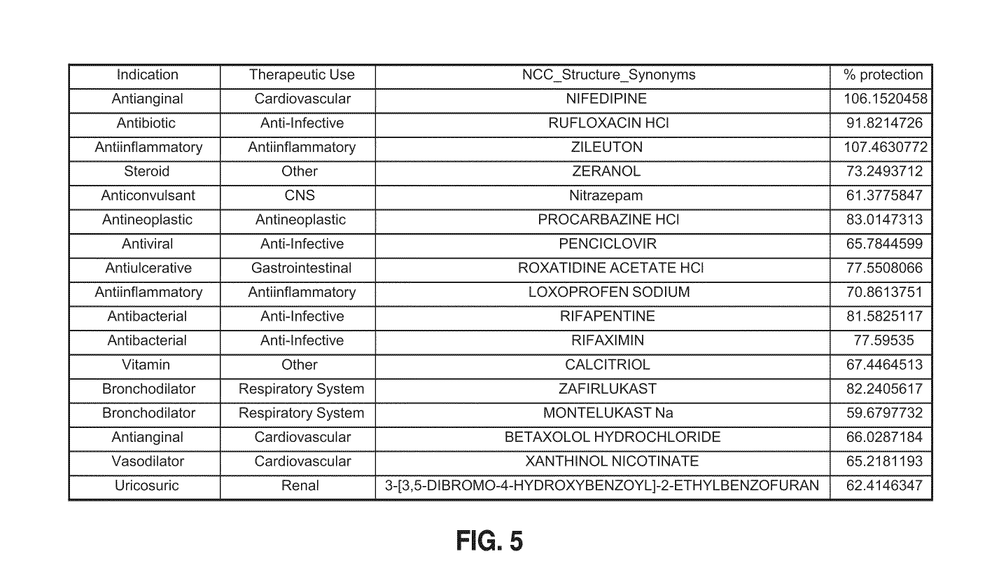

Alternatively, or in addition, the small molecule compound for reducing 5-lipoxygenase activity can be any of those listed in FIG. 5 of this application. For example, the small molecule compound for selectively inhibiting 5-lipoxygenase in the methods described herein can be racemic Zileuton (ZYFLO®), known as ±-[1-(1-benzothien-2-yl)ethyl]-N-hydroxyurea. Zileuton is a hydroxylurea having a benzothienylethyl group and has been used for the treatment of asthma (see U.S. Patent Application 2011/0077305, hereby incorporated by reference).

The chemical structure and methods for synthesizing racemic Zileuton are described in U.S. Pat. Nos. 4,873,259 and 6,080,874, each of which is hereby incorporated by reference, particularly with regard to those sections showing the formula of Zileuton (±-[1-(1-benzothien-2-yl)ethyl]-N-hydroxyurea) and the method for preparing Zileuton. Accordingly, the small molecule compound or composition for selectively inhibiting 5-lipoxygenase in the methods described herein can be any of those benzo[b]thienyl substituted N-hydroxyureas described in U.S. Pat. No. 6,080,874, which is hereby incorporated by reference. See in particular the compounds defined by claims 1-3 of U.S. Pat. No. 6,080,874. More specifically, the racemic Zileuton used in any of the present methods can have the formula:

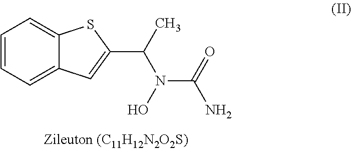

Alternatively, an enantiomerically pure form of Zileuton, such as (R)-Zileuton, may be useful in the present method to inhibit 5-lipoxygenase. The chemical structure of (R)-Zileuton is described in U.S. Patent Application Publication 2010/0273868, which is hereby incorporated by reference. See in particular pages 1-3 of U.S. 2010/0273868, describing the chemical structure of Zileuton and methods of synthesizing racemic and (R)-Zileuton. The Zileuton used in the present methods may have the formula defined by any of claims 1-12 of U.S. Pat. No. 4,873,259, which is hereby incorporated by reference.

Additionally, U.S. Patent Application Publication 2009/0227634, which is hereby incorporated by reference, describes several small molecule pyrazole derivatives that reportedly may be used to inhibit or antagonize 5-LO activity. See in particular the list of small molecules defined by claims 1-5 in U.S. Patent Application Publication 2009/0227634. For example, the small molecule compound can be (2S,4R)-4-(2-fluoro-3-{[4-(1-methyl-1H-pyrazol-5-yl)phenyl]thio}phenyl)-2-methyltetrahydro-2H-pyran-4-carbonitrile having the formula:

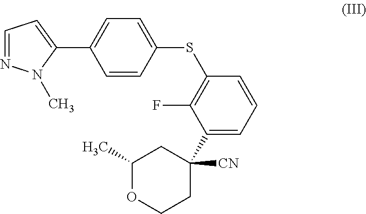

### (2S,4R)-4-(2-fluoro-3-{[4-(1-methyl-1H-pyrazol-5-yl)phenyl]thio}phenyl)-2-methyltetrahydro-2H-pyran-4-carbonitrile

Additionally, Hofmann et al. ((2011) J. Med. Chem. 54, 1943-1947) teaches that 5-benzylidene-2-phenylthiazolinones act as potent direct 5-LO inhibitors and teaches a core structure, or thiazolinone scaffold, common to this class of inhibitors. Thus Hofmann et al. teach 5-LO inhibitors having the core formula:

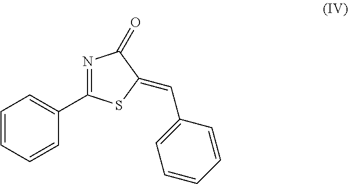

More specifically, Hofmann et al. show a class of 5-LO inhibitors having the formula:

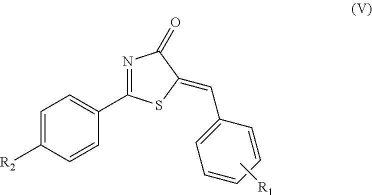

### Where R₁ is O—CH₃, OH, or Cl; and R₂ is Cl, CH₃, or H.

In addition, U.S. Pat. No. 7,829,535, which is hereby incorporated by reference, describes several small molecules that reportedly reduce 5-lipoxygenase activity, which compounds may be useful for treating AMD. See especially column 23, line 51 through column 25, line 13, and claims 13-16 in U.S. Pat. No. 7,829,535, hereby incorporated by reference, for a list of such compounds. Thus, for example, U.S. Pat. No. 7,829,535 teaches that a 5-lipoxygenase inhibitor can be 3-[1-(4-chlorobenzyl)-3-t-butyl-thio-5-isopropylindol-2-yl]-2,2-dimethylpropanoic acid (MK886); 3-(1-(4-chlorobenzyl)-3-(1-butyl-thio)-5-(quinolin-2-yl-methoxy)-indol-2-yl)-2,2-dimethyl propanoic acid) (MK-591); nordihydroguaiaretic acid (NDGA); 2-(12-hydroxydodeca-5,10-diynyl)-3,5,6-trimethyl-1,4-benzoquinone (AA861); or (N-(1-benzo[b]thien-2-ylethyl)-N-hydroxyurea) (Zileuton). U.S. Pat. No. 7,829,535 further teaches that 5-lipoxygenase inhibitors include tenidap, zileuton, flobufen, lonapalene, tagorizine, Abbott A-121798, Abbott A-76745, Abbott A-78773, Abbott A-79175, Abbott ABT 761, Ciba-Geigy CGS-26529, Biofor BF-389, Cytomed CMI-392, Leo Denmark ETH-615, lonapalene, Merck Frosst L 699333, Merckle ML-3000, 3M Pharmaceuticals R-840, linazolast (TMK-688), Zeneca ZD-7717, Zeneca ZM-216800, Zeneca ZM-230487, and Zeneca ZD-2138.

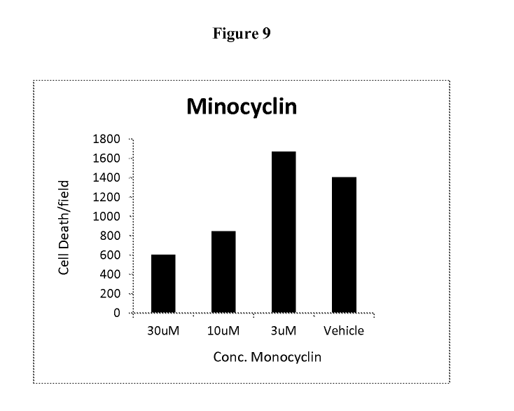

In addition, U.S. Pat. No. 6,455,541, which is hereby incorporated by reference, teaches the small molecule 5-LO inhibitors NGDA, MK886, and ZM230,487. See in particular FIGS. 9-11 in U.S. Pat. No. 6,455,541, hereby incorporated by reference.

In addition, U.S. Pat. No. 4,634,766, which is hereby incorporated by reference, teaches a class of small molecule 1,4-diaza-phenothiazines that are said to be inhibitors of mammalian 5-lipoxygenase. See in particular the compounds defined by claims 1-8 in U.S. Pat. No. 4,634,766, hereby incorporated by reference.

In addition, U.S. Pat. No. 4,833,164, which is hereby incorporated by reference, describes 2-substituted-1-naphthols and their use as 5-lipoxygenase inhibitors. See especially claims 1-19 therein, describing naphthol compounds and compositions thereof for inhibiting 5-lipoxygenase.

U.S. Pat. No. 5,093,356, which is hereby incorporated by reference, teaches indenyl hydroxamic acids and hydroxylureas as inhibitors of 5-lipoxygenase. See in particular the small molecule compounds defined by claims 1-5.

Compounds that reduce 5-LO activity may be used individually or in combination. When used in combination, the compounds may be administered together or separately at intervals, with or without resting periods between administrations.

Uses, Formulations, and Administration

Compounds that reduce the activity of 5-lipoxygenase are useful for the treatment of age related macular degeneration (AMD). More specifically, compounds that inhibit 5-lipoxygenase may be used to treat atrophic or dry AMD and neovascular or wet AMD.

The present invention is also directed to the preparation of a medicament, or pharmaceutical composition, for the treatment AMD. The medicament contains a pharmaceutical acceptable composition, which comprises a therapeutically effective amount of a compound that inhibits 5-LO, as described herein, together with a pharmaceutical acceptable carrier. The medicament or pharmaceutical composition may further contain a second or additional therapeutic agent for the treatment of AMD.

These pharmaceutical compositions can be used as a medicament and administered to a subject suffering from AMD, such as a human or non-human mammal, in need of treatment of AMD. Different types of suitable dosage forms and medicaments are well known in the art, and can be readily adapted for delivery of 5-LO inhibitory compounds of the present invention. Methods for administering 5-LO inhibitory compounds and compositions thereof to the subject in need of treatment of AMD include, but are not limited to, oral, systematic, parenteral, local, and topical delivery. The dosage forms can be tablets, capsules, intravenous injections, intramuscular injections, local injections, topical creams, gels and ointments, eye drops, ophthalmic solutions, ophthalmic suspensions, ophthalmic emulsions, intravitreal injections, subtenon injections, ophthalmic bioerodible implant, and non-bioerodible ophthalmic inserts or depots, nasal sprays and ointment, various rectal or vaginal preparations.

Accordingly, compounds and pharmaceutical compositions that selectively or indirectly inhibit 5-LO can be administered systemically, orally, subcutaneously, intravenously, intraperitoneal, intravitreal, retrobulbar, with a bioerodible implant drug delivery system into the eye of a subject, or topically to the eye, or by injecting a biodegradable drug delivery system containing a 5-LO inhibitor into the eye of the subject such as the vitreous cavity of the eye.

Bioerodible or biodegradable ocular implants and biodegradable (e.g., PLA and/or PLGA-containing) polymer drug delivery systems that may be useful for administration of a 5-lipoxygenase inhibitory compound in the present methods are described in U.S. Patent Application Publication 2010/0015158, which is hereby incorporated by reference. See in particular pages 7-9 (paragraphs 71-94) in US 2010/0015158.

Thus, one embodiment of the present invention is an intraocular biodegradable drug delivery system comprising a 5-lipoxygenase inhibitor (i.e., a compound that reduces 5-lipoxygenase activity) and a biodegradable polymer such as a poly(lactide) (PLA) polymer or a poly(lactide-co-glycolide) (PLGA) polymer or a combination of PLA and PLGA polymers. The drug delivery system can be injected or implanted into an intraocular location to provide sustained release of the 5-LO inhibitor. Polylactide (PLA) polymers exist in two chemical forms, poly(L-lactide) and poly(D,L-lactide). A PLGA is a co-polymer that combines poly(D,L-lactide) with poly(glycolide) in various possible ratios. The higher the glycolide content in a PLGA the faster the polymer degradation.

In one embodiment of the invention, a drug delivery system for intraocular administration (i.e. by intravitreal implantation or injection) of a 5-LO inhibitor comprises, consists of, or consists essentially of a 5-LO inhibitor and at least a 75 weight percent of a PLA and no more than about a 25 weight percent of a poly(D,L-lactide-co-glycolide) polymer.

Also included within the scope of the present invention are suspensions of microspheres incorporating a 5-LO inhibitor suspended in a hydrogel (such as a polymeric hyaluronic acid), which can be administered to an intraocular location through a syringe needle.

Thus, another embodiment of the present invention includes using a biodegradable drug delivery system comprising a 5-LO inhibitor in the eye of a subject (e.g., a human individual or non-human mammal or other animal) suffering from AMD or related ocular condition to relieve, reduce, or protect against one or more symptoms associated with age related macular degeneration (AMD) or related ocular condition.

One embodiment of the present invention is a method comprising placing a biodegradable drug delivery system comprising a 5-LO inhibitor and a biodegradable polymer or polymer mixture (e.g., PLA or PLGA or both) in the eye of an individual suffering from AMD or at risk of developing AMD. The individual may be a human or non-human animal, including but not limited to a rabbit, mouse, horse, dog, rat, or monkey.

The 5-LO inhibitory compound may be combined and delivered together with a second therapeutic agent for the treatment of AMD.

### DEFINITIONS

The term “Age-related macular degeneration” or “AMD” is given the meaning understood by those or ordinary skill in the art.

As used herein, “small molecule compounds” and “small molecules” do not include polypeptides or polynucleotides and are preferably less than about 1000 kDa.

Therapeutic levels” or “therapeutic amount” means an amount or a concentration of an active agent that has been locally delivered to an ocular region that is appropriate to treat an ocular condition such as AMD so as to reduce or prevent a symptom of an ocular condition such as AMD.

“Treating” means to administer a treatment to a human or non-human subject to obtain a therapeutic effect. Treating may include reducing or slowing a clinical symptom associated with an ocular condition such as AMD, including but not limited to vision loss and/or neovascularization.

### EXAMPLES

### Example 1

### Development of Complement Assay

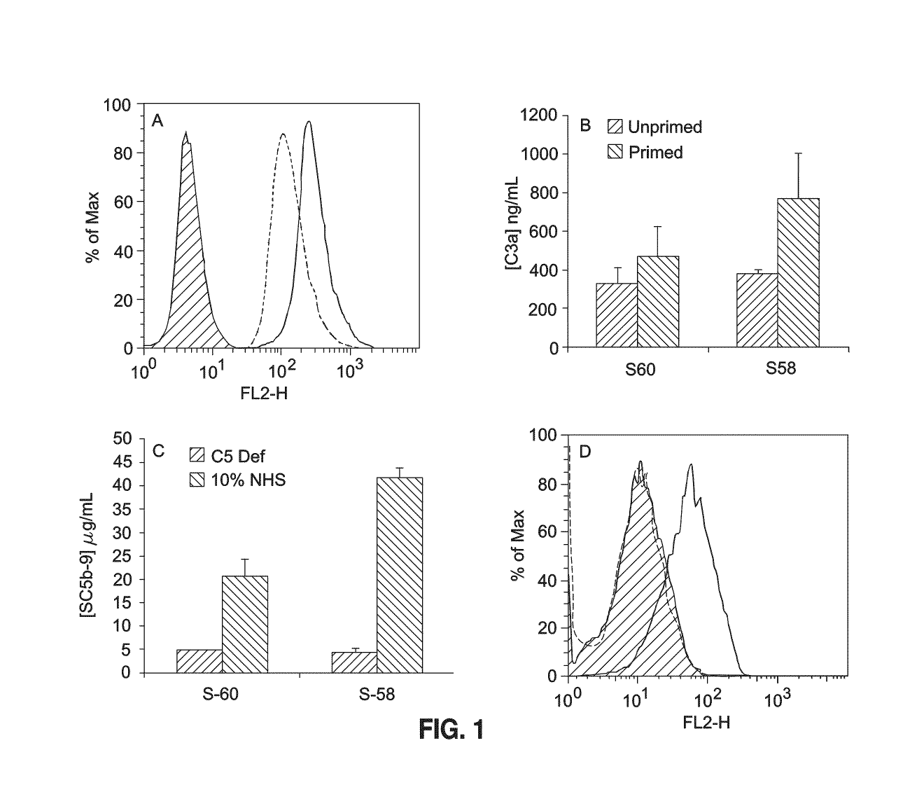

Sheep were immunized with intact ARPE-19 cells and anti serum collected and purified over and IgG affinity column. Two anti serums were developed S-58 and S-59. The Anti-serum against ARPE-19 (5-58 and S-60) recognize ARPE-19 cells (FIG. 1A). Of the two, only S-58 leads to efficient complement activation (FIGS. 1B and 1C) and deposition of MAC on the cell surface as determined by FACS analysis (FIG. 1D).

### Example 2

### Validation of Complement Attack and Cell Death

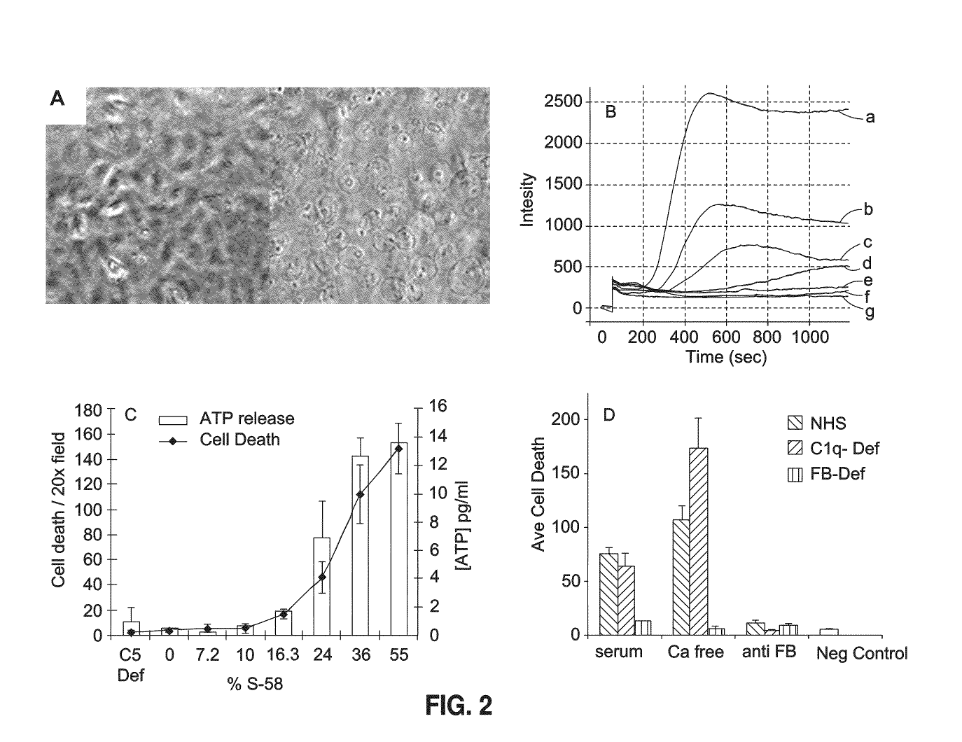

Initiation of complement attack by 5-58 on ARPE-19 cells induced swelling (FIG. 2A), a dose dependent rise in intracellular calcium (FIG. 2B) as well as cell death and ATP release into the supernatant (FIG. 2C). Blocking the alternative pathway, but not the classical pathway, inhibited cell death (FIG. 2D).

### Example 3

### Synergistic Relationship Between Oxidative Stress and Complement

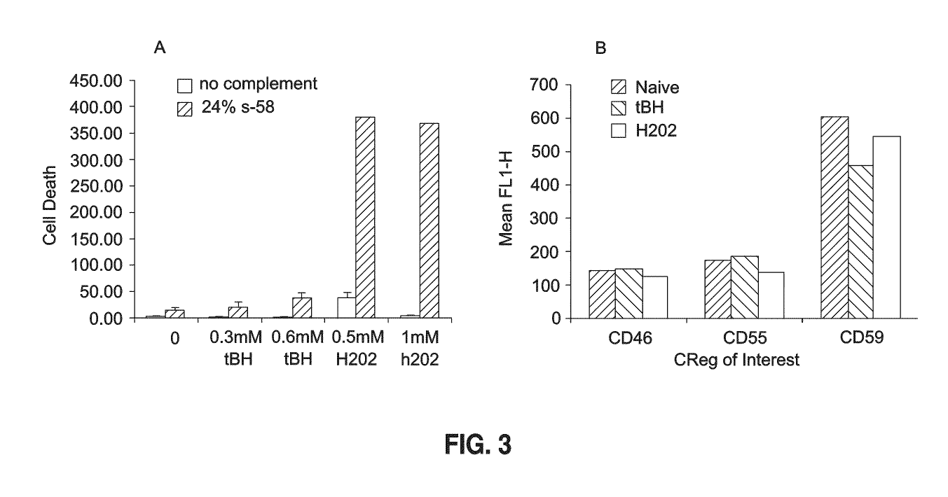

Oxidative stress and complement activation are the two most highly cited factors associated with occurrence and progression of AMD. To examine functional consequences of this relationship we treated ARPE-19 cells with either H₂O₂ or t-BH followed by challenge with complement. Oxidative stress induced by t-BH caused a 20-30% increase in cell death while that of H₂O₂ resulted in a >10 fold increase (FIG. 3A). FACS analysis of ARPE-19 after treatment with t-Bh or H₂O₂ indicated minimal impact in expression levels (FIG. 3B).

### Example 4

### Library Screen

ARPE-19 cells were plated at sub confluence in 96 well culture plates and cultured 24 to 48 hrs. The cells were then treated with 1:1000 dilution of each compound from the NIH clinical collection library overnight. The following day culture media was collected and set aside. The cells were washed and then treated with 0.77 mM H₂O₂ in hanks buffered saline solution (HBSS) for 90 minutes in the presence of fresh compound from NIH clinical collection library at a 1:1000 dilution. Cells were then washed and primed with a solution of HBSS containing 24% S-58 for 30 minutes. After the priming step cells were washed and then treated with 6% C1q deficient human serum in the original cell culture supernatant collected prior to H₂O₂ treatment. 90 minutes after addition of the human serum cells were labeled with the membrane impermeant nuclear dye SYTOX® orange and the number of positive cells determined. This count was then used to determine level of cell death. Quantitation and imaging were accomplished by capturing a single field per objective using a 10× objective and then counting the number of positive nuclei using metamorph image analysis software. Data was collected and expressed as % protection relative to wells containing vehicle alone (DMSO).

### Summary of Examples 1-4

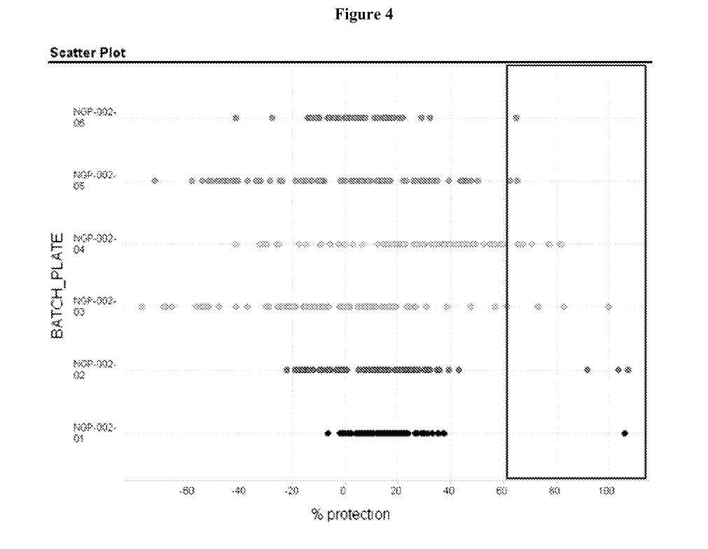

Data was collected and the level of protection by each compound determined by averaging the results of two independent experiments. FIG. 4 is the distribution of results indexed to compound plate (y-axis) and Protection (x-axis). Based on the distribution a break point at 60% and greater protection was chosen as a cutoff for compounds that showed significant protection in the assay (Boxed region in FIG. 4). FIG. 5 contains a list of compounds with protective properties. Of the 17 compounds selected, six compounds (Zeranol, Penciclovir, Rifapentine, Rifaximin, calcitrol, 3-[3,5-DIBROMO-4-HYDROXYBENZOYL]-2-ETHYLBENZOFURAN) were deemed to function as anti-oxidants in the assay. Of the remaining 11 compounds, four fall into a single class associated with modulation of the arachidonic acid pathway. This included Zileuton, Zafirlukast, Montelukast, and Loxoprofen. Significantly three of the four target the 5-lipoxygenase pathway identifying it as the primary target. The targeting was both direct at 5-lipoxygenase (Zileuton) and indirect at the leukotiene receptor (Zafirlukast, Montelukast). These results indicate that blocking the 5-lipoxygenase pathway is a novel therapeutic for treating AMD, especially at the point of synergy between oxidative stress and complement activation.

The observation that 5-lipoxygenase has a direct effect on RPE cells suggests a potential mechanism to protect as well as restore function to RPE cells in age related macular degeneration. This unpredicted and novel discovery provides the repurposing of inhibitors of the leukotriene pathway (both leukotriene synthesis and antagonisms of their receptors) as therapeutics for treatment of age related macular degeneration in mammals, especially humans.

Additionally, the results identify modulators of 5-lipoxygenase as therapeutic targets for AMD, including agents increasing cAMP kinase activity such as phospholiesterase inhibitors type IV, Prostaglandin E2, adenosine a2 receptor agonist, compounds inhibiting 5-lipoxygenase activation protein (FLAP) as represented by MK-0591 and MK-886, compounds modulating PLA2 activation, as well as compounds targeting p38 pathway and Rac activation.

Finally the observation that the COX inhibitor (Loxoprofen) was also identified suggest that dual inhibitors targeting both COX1,2 and 5-lipoxygenase would be beneficial in treating age related macular degeneration.

### Example 5

### The 5-Lipoxygenase Inhibitor MK886 Protects ARPE-19 Cells from Oxidative Stress and Complement Attack

The intervention of a second protein, 5-LO-activating protein (FLAP), is required before 5-LO can become catalytically active. MK 886 binds to FLAP and prevents 5-LO activation. See Abramovitz, M., Wong, E., Cox, M. E., et al. “5-Lipoxygenase-activating protein stimulates the utilization of arachidonic acid by 5-lipoxygenase.” Eur J Biochem 215 105-111 (1993). MK886 (1-[(4-chlorophenyl)methyl]-3-[(1,1-dimethylethyl)thio]-α,α-dimethyl-5-(1-methylethyl)-1H-indole-2-propanoic acid, sodium salt) has the formula:

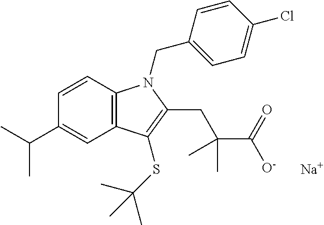

### 1-[(4-chlorophenyl)methyl]-3-[(1,1-dimethylethyl)thio]-α,α-dimethyl-5-(1-methylethyl)-1H-indole-2-propanoic acid, sodium salt

The observation that inhibition of 5-lipoxygenase is protective against the combined effects of oxidative stress and complement attack in ARPE-19 cells prompted us to test the 5 lipoxygenase activating protein inhibitor MK886 for its ability to protect ARPE-19 cells from oxidative stress and complement attack in vitro. The cell protection assay was carried out essentially as described in Examples 1-4.

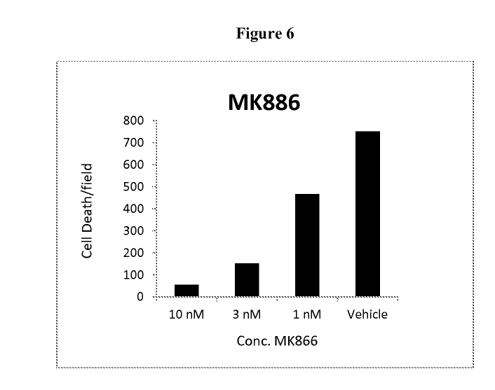

As shown by FIG. 6, treatment of cells with MK886 following oxidative stress and complement attack (according to the method of Examples 1-4) effectively blocked cell death in a dose dependent manner as compared to the vehicle (DMSO) alone, indicating the significance of the 5-lipoxygenase pathway, and showing that reducing 5-LO activity by inhibiting FLAP can also be used as a method to protect ARPE-19 (retinal pigment epithelial) cells from oxidative stress and complement attack.

### Example 6

### Comparison of 5-LO and 15-LO Inhibitors as Protective Agents

In addition to the MK886 inhibitor we also evaluated other know 5-lipoxygenase inhibitors, including 2-TEDC and monocyclin, for their ability to protect ARPE-19 cells against oxidative stress and complement attack, using the cell protection assay described in Examples 1-4.

2-TEDC (2-(1-Thienyl)ethyl 3,4-dihydroxybenzylidenecyanoacetate) has the formula:

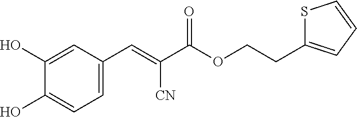

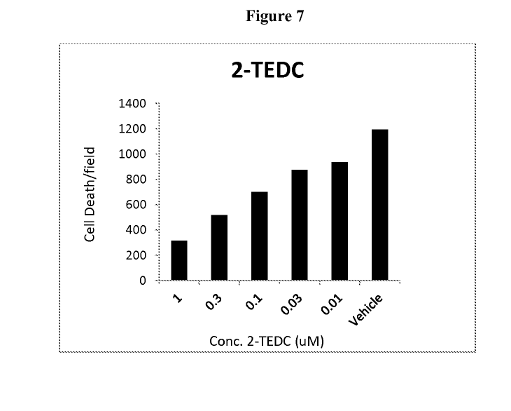

As shown by FIG. 7, 2-TEDC blocked cell death within the assay system with an I.C.50 of 0.1, which correlates with the known I.C.50 (0.09 um) inhibition of 5-lipoxygenase by this compound (Cho, H., et al., Novel caffeic acid derivatives: extremely potent inhibitors of 12-lipoxygenase. J Med Chem, 1991. 34(4): p. 1503-5). This does not appear to involve either the 12 lipoxygenase or 15 lipoxygenase activity of the compound, since little activity is seen at the known I.C. 50 for 12 lipoxygenase and we observed nearly 100% protection at the known I.C.50 for 15 lox (Cho, H., et al., Novel caffeic acid derivatives: extremely potent inhibitors of 12-lipoxygenase. J Med Chem, 1991. 34(4): p. 1503-5).

To further rule out the involvement of 15 lox we used the 15 lipoxygenase inhibitor PD 146176 (6,11-dihydro-[1]benzothiopyrano[4,3-b]indol) (Bocan, T. M., et al., A specific 15-lipoxygenase inhibitor limits the progression and monocyte-macrophage enrichment of hypercholesterolemia-induced atherosclerosis in the rabbit. Atherosclerosis, 1998. 136(2): p. 203-16).

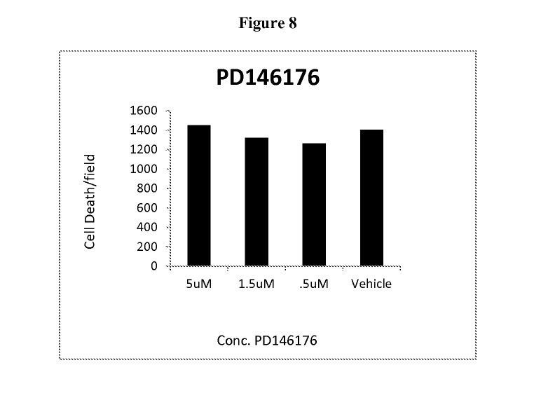

As shown in FIG. 8, no protection against cell death was observed with PD146176, indicating that 5-lipoxygenase is the key target for protecting retinal cells against complement and oxidative stress mediated cell death and ruling out the involvement of 12 and 15 lipoxygenases. To further examine the contribution of 5-lipoxygenase we tested a third commercially available compound known to inhibit 5-lipoxygenase activity, Minocyclin (Song, Y., et al., “Minocycline protects PC12 cells from ischemic-like injury and inhibits 5-lipoxygenase activation”Neuroreport, 2004. 15(14): p. 2181-4). As shown in FIG. 9, minocyclin, like MK886 and 2-TEDC, dose dependently protected against complement and oxidative stress mediated cell death.

### Example 7

### In Vivo Models

### 5-LO Inhibitors Protect Against Functional Loss of Retinal ERG A and B-Wave Amplitudes In Vivo Following Oxidative Stress Induced by Intravitreal PQ Injection

To further explore the protective effects of inhibition of the 5-lipoxygenase pathway, we examined the ability of 5-lipoxygenase inhibition in two in vivo models. The first in vivo model was a model of retinal oxidative stress induced by intravitreal injection of Paraquat in SOD1−/+ animals (Dong, A., et al., Superoxide dismutase 1 protects retinal cells from oxidative damage. J Cell Physiol, 2006. 208(3): p. 516-26). In addition to elevation of oxidative stress Paraquat is also known to induce activation of complement (Sun, S., et al. (2011 Mar. 18) “Complement Inhibition Alleviates Paraquat-Induced Acute Lung Injury” Am J Respir Cell Mol Biol. EPub), allowing us to examine effects of oxidative stress and complement activation in vivo similar to that observed in the in vitro RPE assay, above. The second in vivo model in which we examined the effects of 5-lipoxygenase inhibition was a rodent light induced retinal degeneration model. This model of light damage is used for modeling the pathology of age related macular degeneration AMD and testing of AMD therapeutic compounds (Marc, R. E., et al., Extreme retinal remodeling triggered by light damage: implications for age related macular degeneration. Mol V is, 2008. 14: p. 782-806; Collier, R. J., et al. (2011 Apr. 5. Epub ahead of print) Complement Deposition and Microglial Activation in the Outer Retina in Light-Induced Retinopathy: Inhibition by a 5-HT1A agonist. Invest Ophthalmol V is Sci

The 5-LO inhibitory compound used in these in vivo studies was PF-4191834, a selective non-redox 5-lipoxygenase inhibitor. (Masferrer, J. L., et al., J Pharmacol Exp Ther. 334(1): pp. 294-301).

PF-4191834 has the formula:

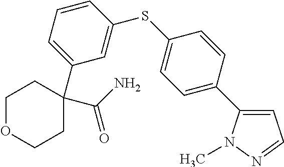

### 4-(3-(4-(1-Methyl-1H-pyrazol-5-yl)phenylthio)phenyl)-tetrahydro-2H-pyran-4-carboxamide

### Materials and Methods

### Example 7

### Intravitreal Paraquat Injection

SOD−/+ C57BL/6 mice received first and second oral doses of 1 mg/kg of PF-4191834 sixteen (16) hrs and 4 hrs prior to the paraquat injection and then a third oral dose of 1 mg/kg of PF-4191834 four (4) hours after the paraquat injection. Control animals received equivalent volume of vehicle (0.5% Avicel R-91 and 0.1% Tween-80 in dH₂O) to compound treated animals.

Animals were anesthetized with ketamine (75 mg/kg) and dexmedetomidine (1.0 mg/kg), i.p. Both eyes were kept moist with 1-2 drops of saline. The eye being injected was initially treated with two drops of 0.5% proparacaine. The eye lids were gently rubbed together to insure maximal distribution and exposure of the eye surface to the anesthetic. The eye was then proptosed using a proptosing instrument, exposing the anterior surface of the eye. Using a 36 gauge needle attached to a Hamilton syringe and micromanipulator the needle was inserted posterior to the ora serrata and into the intravitreal space. A 1 μl injection of 0.75 mM Paraquat was made using a Hamilton Lab animal injector (LASI 115) designed to deliver between 25 nanoliters to 25 microliters per injection. Injections were performed under a dissection microscope and a foot pedal to execute injections. Animals were revived by administration of 0.5 mg/kg atipamezole i.p., returned to their cages, and warmed using a heating pad and monitored until awake.

### Electroretinogram (ERG):

The ERG was performed 1 day post paraquat injection. Scotopic ERGs were performed using a 0.001, 0.01, and 1 cd·s/m2 flash stimulus and 20 Hz flicker protocol. Mice were dark-adapted for a 12 hour period and ERG recordings were performed using the Espion ERG Diagnosys system. Pupils were first dilated with drops of both 1% AK-Pentolate (Cyclopentolate Hydrochloride) and 10% AK-Dilate (Phenylephrine Hydrochloride). After the pupils were dilated (approximately 5 minutes), mice were anesthetized with a ketamine (75 mg/kg) and dexmedetomidine (1.0 mg/kg) cocktail (injected i.p.). Celluvisc was placed on the eyes followed by surface electrodes. The ERG was then recorded with the animal on a heating pad (takes 7-8 minutes). Animals were revived by administration of 0.5 mg/kg atipamezole (injected i.p.), returned to their cages, and warmed using a heating pad and monitored until awake.

### Blue Light Exposure:

Balb/c mice were dosed orally (PO) at 1 mg/Kg starting 16 hours prior to blue light, and then BID for the next 14 days. Control animal received an equivalent volume of vehicle (0.5% Avicel R-91 and 0.1% Tween-80 in dH₂O) to compound treated animals.

Animals were first dark adapted for 16 hours prior to blue light. Animals were then exposed to blue light at 4800 lux for 8 hours. Animals were then moved to normal housing conditions.

### Electroretinogram (ERG):

The ERG was performed 2 weeks post blue light (4 days post OCT to allow for cataracts to clear). Scotopic ERGs were performed using a 0.001, 0.01, and 1 cd·s/m2 flash stimulus and 20 Hz flicker protocol. Mice were dark-adapted for a 12 hour period and ERG recordings were performed using the Espion ERG Diagnosys system. Pupils were first dilated with drops of both 1% AK-Pentolate (Cyclopentolate Hydrochloride) and 10% AK-Dilate (Phenylephrine Hydrochloride). After the pupils were dilated (approximately 5 minutes), mice were anesthetized with a ketamine (75 mg/kg) and dexmedetomidine (1.0 mg/kg) cocktail (injected i.p.). Celluvisc was placed on the eyes followed by surface electrodes. The ERG was then recorded with the animal on a heating pad (takes 7-8 minutes). Animals were revived by administration of 0.5 mg/kg atipamezole (injected i.p.), returned to their cages, and warmed using a heating pad and monitored until awake.

### Tissue Harvest:

Mice were euthanized with CO₂, decapitated (to facilitate exsanguination of the periorbital region), eyes excised, and then marked along their superior aspect with tissue marking dye.

Eyes were immersion fixed over night at room temperature in Davidson's Fix special (BBC Biochemical, Mt Vernon, Wash.), dehydrated for 1 hr in 50% EtOH at room temperature, and then placed into 70% EtOH at room temperature. Eyes were stored in 70% EtOH until subjected to automated paraffin processing. After processing, eyes were embedded in paraffin, sectioned on a rotary microtome at 5 μm thickness through the optic nerve head parallel to the vertical meridian, and mounted on glass slides. Sections were baked onto slides, subjected to automated H&E staining, and coverslipped using permanent mounting medium. Stained tissue sections were then examined by an observed blinded to treatment regiments and scored to determine the level of damage to the outer nuclear layer.

### Results

### Example 7

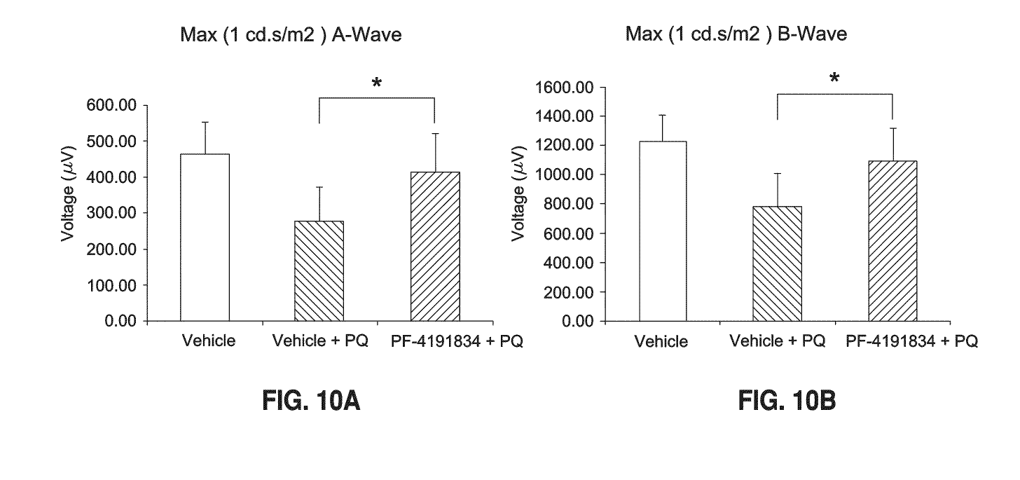

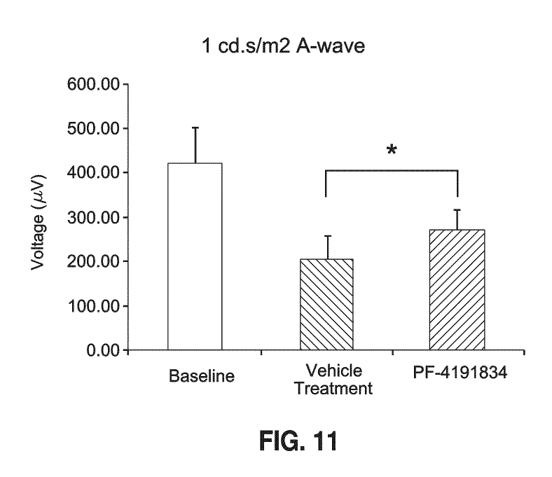

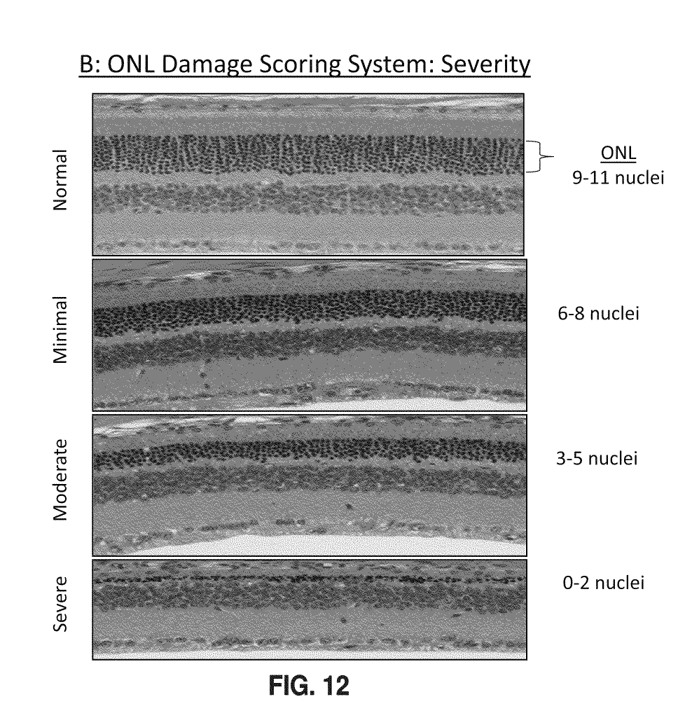

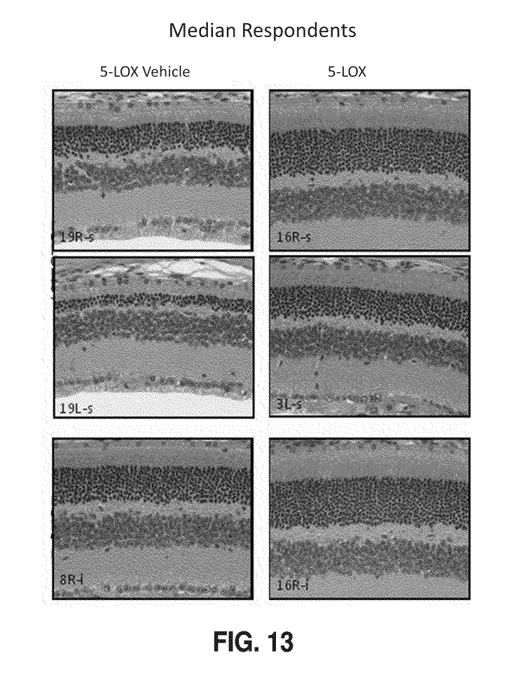

As shown in FIGS. 10A and B, inhibition of 5-lipoxygenase by administration of PF-4191834 significantly protected against functional loss of retinal ERG A and B-wave amplitudes in the paraquat induced retinal oxidative stress model. As shown by FIG. 11, the 5-lipoxygenase inhibitor PF-4191834 also protected against the loss of ERG A-wave amplitude in retinal cells induced by light damage in an animal model, similar to FIG. 10. We also observed that treatment of animals with PF-4191834 protected retinal structure and preserved photoreceptors as indicated by the retention of thickness in the outer nuclear layer (see FIGS. 12, 13 and Table 2).

TABLE 2
Summary of outer nuclear layer morphology relative to non-light
damaged (normal ONL). Data are presented as % change
relative to normal non pathological retinas.
Frequency of normal ONL

| Treatment | Superior Retina | Inferior Retina |
|---|---|---|
| Vehicle | 2/6 (33%) | 2/6 (33%) |
| PF-4191834 | 5/10 (50%) | 8/10 (80%) |

What is claimed is:

1. A method of treating age related macular degeneration in a subject in need thereof, comprising administering to said subject a therapeutically effective amount of a compound that reduces 5-lipoxygenase (5-LO) activity.

2. The method of claim 1, wherein the compound that reduces 5-lipoxygenase activity is a small molecule.

3. The method of claim 2, wherein the compound that reduces 5-lipoxygenase activity is PF-4191834.

4. The method of claim 1, wherein the compound that reduces 5-lipoxygenase activity reduces 5-lipoxygenase activity by inhibiting 5-lipoxygenase activating protein (FLAP).

5. The method of claim 4, wherein the compound that inhibits FLAP is a small molecule.

6. The method of claim 5, wherein the compound that inhibits FLAP is MK886.

7. The method of claim 1, wherein the compound that reduces 5-lipoxygenase activity is Zileuton.

8. The method of claim 1, wherein the compound that reduces 5-lipoxygenase activity is administered in the form of a pharmaceutically acceptable composition comprising a pharmaceutically acceptable diluent.

9. The method of claim 1, wherein compound that reduces 5-lipoxygenase activity is administered together with a second therapeutic agent for treating AMD.

10. An intraocular biodegradable drug delivery system comprising a compound that reduces 5-lipoxygenase activity and a biodegradable polymer, wherein the biodegradable polymer is a poly(lactide) (PLA) polymer, a poly(lactide-co-glycolide) (PLGA) polymer, or a combination of PLA and PLGA polymers.
